(19) United States
(12) Patent Application Publication
(10) Pub. No.: US 2004/0102365 A1
(43) Pub. Date: May 27, 2004

(54) Pharmaceutical composition for prevention and remedy of osteoporosis

(76) Inventor: Kazuo Nagai, Kanagawa (JP)

Correspondence Address: BIRCH STEWART KOLASCH & BIRCH, PO BOX 747, FALLS CHURCH, VA, 22040-0747, US

(21) Appl. No.: 10/467,802

(22) PCT Filed: Feb. 14, 2001
(86) PCT No.: PCT/KR01/00220

(51) Int. Cl. C07K 7/64; A61K 38/12

(52) U.S. Cl. 514/10; 530/317

(57) ABSTRACT

The present invention relates to a pharmaceutical composition for prevention and remedy of osteoporosis containing destruxin derivative as an active principle and cyclodepsipeptide of this invention, originated mold, is greatly efficacious on the activity inhibition ability for the osteoclast without regard to existence of the parathyroid hormone.

### TECHNICAL FIELD

The present invention relates to a pharmaceutical composition for the prevention and treatment of osteoporosis, and more particularly, to a pharmaceutical composition containing a destruxin derivative as an active ingredient, which is effective for the prevention and treatment of osteoporosis.

### PRIOR ART

In general, osteoporosis, which is the most common among bone-related metabolic diseases, is caused by reduction in bone mineral density with advancing age. Osteoporosis has become a serious social problem in many advanced countries, triggering many attempts to treat osteoporosis. Especially, in Korea, which is an increasingly aged society, osteoporosis is becoming a common age-related disease. Also, in America, the prevention and treatment of osteoporosis is a severe social problem to be solved, resulting from the fact that about ten million people suffer from osteoporosis and about eighteen million people have low bone mineral density. One in two females and one in eight white males in America are expected to experience bone fractures in their life, and moreover, over two million men in America are already afflicted with osteoporosis, and ninety thousands men in America suffer from pelvis fractures every year, with about one-third of them dying less than one year thereafter. Demand for treatment agents of osteoporosis continues to increase rapidly, by about 10 percent each year in European advanced countries, which have also joined the ranks of aged societies.

Bone tissue is a dynamic tissue where bone is remolded through continuous cycles of osteoblast-mediated bone formation and osteoclast-mediated bone resorption. The mechanism of bone formation and resorption still remains uncertain, but it is well known that an imbalance in bone formation and bone resorption rates leads to osteoporosis, in which bone resorption is accelerated while bone formation is inhibited. Based on this finding, there have been many research attempts to develop bone resorption-inhibiting agents having strong inhibitory activity versus osteoclasts.

On the other hand, destruxin derivatives, cyclodepsipeptides isolated from fungus including Melarhizium anisopliae that inhabits insects, are well known to have insecticidal activity as well as antiviral activity and toxicity for leukemia cells (F. Cavelier et al., J. Peptide Res., 50, 1997, 94-101), and can be used as cardiotonics or inducers of erythropoietin. In addition, it has been discovered that destruxin derivatives have insecticidal activity through inhibition of the activity of Na⁺-ATPase enzyme. However, until now, there has no report of destruxin derivatives effective for preventing and treating osteoporosis.

In bone metabolism, calcified bone is associated with bone resorption, and osteoclasts, which are attached to the surface of bone, mediates bone resorption under the infuence of certain hormones, while producing cell debris, which is subsequently removed (Ross et al., 1995). Among the hormones, parathyroid hormone (PTH) induces the formation of osteoclasts, which promote resorption of bone matrix, and activates osteoclasts, resulting in the release of calcium.

In addition, osteoclasts, which are associated with appearance of osteoporosis, secrete strong acids into an extracellular compartment formed between osteoclast and bone surface to degrade bone through action of H⁺-ATPase enzyme, leading to acidification of the osteoclast-bone interface.

Based on the fact that the two enzymes, Na⁺-ATPase and H⁺-ATPase, belong to the vacuolar ATPase family, in spite of being different in genetic level, the intensive and thorough research for destruxin derivatives, leading to the present invention, resulted in the finding that destruxin derivatives have an excellent inhibitory activity against osteoclast proliferation as well as osteoclast activation.

Accordingly, it is an object to provide a pharmaceutical composition for the prevention and treatment of osteoporosis containing a destruxin derivative having inhibitory activity against osteoclasts.

### DISCLOSURE OF THE INVENTION

In accordance with an aspect of the present invention, there is provided a pharmaceutical composition containing a destruxin derivative as an active ingredient, and the destruxin derivative is represented by the chemical formula I:

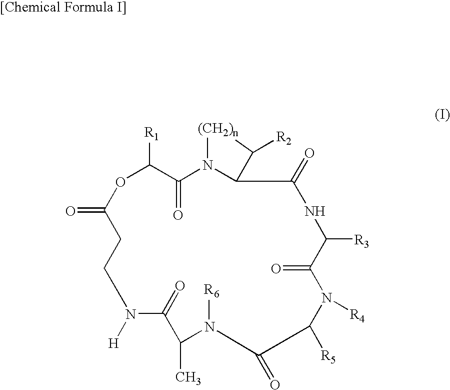

wherein, R₁ group is CH₃, CH₂—CH═CH₂, CH₂CH(CH₃)₂, CH₂CH(CH₃)CH₂OH,

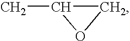

CH₂CH(CH₃)COOH, CH₂CH(OH)CH₂Cl, CH₂—C≡CH, or CH₂CH(OCOCH₃)CH₂Cl; R₂, R₄ and R₆, which may be the same or different, are H or CH₃, R₃ and R₅, which may be the same or different, are CH(CH₃)CH₂CH₃ or CH(CH₃)₂; and n is 2 or 3.

The destruxin derivative can be prepared according to Method 1 described in methods known in the art (Phytochemistry, Vol. 20, pp. 715-723, 1981; J. Chem. Soc. Perkin. Trans. I, 2347-2357, 1989 ; J. Peptide Res. 50, 1997, 94-101; J. Antibiotics, Vol. 50, 1007- 1013, 1997; J. Nat. Prod., 61, 290-293, 1998, etc.). In the known methods, there are disclosed methods of preparing various destruxin derivatives including destruxin A, B, C, D, E, A₁, A₂, B₁, B₂, C₂, D₁, D₂, E₁ and E₂ as well as roseotoxin B, roseocardine, desmethyldestruxin B, and other destruxin derivatives in which the R₁ group is substituted with chlorohydrin, acetylchlorohydrin, etc., and isolation methods and use thereof. The destruxin derivative of the present invention can be used as a pharmaceutically acceptable salt, which may be typically an inorganic or organic salt.

Destruxin derivatives useful in the present invention are given in Table 1, below.

TABLE 1
Destruxin Derivatives

| Destruxin | | | | | | | |
|---|---|---|---|---|---|---|---|
| Derivative | R₁ | R₂ | R₃ | R₄ | R₅ | R₆ | n |
| A | CH₂—CH═CH₂ | H | CH(CH₃)CH₂CH₃ | CH₃ | CH(CH₃)₂ | CH₃ | 2 |
| B | CH₂—CH(CH₃)₂ | H | CH(CH₃)CH₂CH₃ | CH₃ | CH(CH₃)₂ | CH₃ | 2 |
| C | CH₂CH(CH₃)CH₂OH | H | CH(CH₃)CH₂CH₃ | CH₃ | CH(CH₃)₂ | CH₃ | 2 |
| D | CH₂CH(CH₃)COOH | H | CH(CH₃)CH₂CH₃ | CH₃ | CH(CH₃)₂ | CH₃ | 2 |
| E | 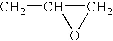 | H | CH(CH₃)CH₂CH₃ | CH₃ | CH(CH₃)₂ | CH₃ | 2 |
| A₁ | CH₂CH═CH₂ | H | CH(CH₃)CH₂CH₃ | CH₃ | CH(CH₃)₂ | CH₃ | 3 |
| A₂ | CH₂CH═CH₂ | H | CH(CH₃)₂ | CH₃ | CH(CH₃)₂ | CH₃ | 2 |
| B₁ | CH₂CH(CH₃)₂ | H | CH(CH₃)CH₂CH₃ | CH₃ | CH(CH₃)₂ | CH₃ | 3 |
| B₂ | CH₂CH(CH₃)₂ | H | CH(CH₃)₂ | CH₃ | CH(CH₃)₂ | CH₃ | 2 |
| C₂ | CH₂CH(CH₃)CH₂OH | H | CH(CH₃)₂ | CH₃ | CH(CH₃)₂ | CH₃ | 2 |
| D₁ | CH₂CH(CH₃)COOH | H | CH(CH₃)CH₂CH₃ | CH₃ | CH(CH₃)₂ | CH₃ | 3 |
| D₂ | CH₂CH(CH₃)COOH | H | CH(CH₃)₂ | CH₃ | CH(CH₃)₂ | CH₃ | 2 |
| E₁ | 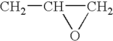 | H | CH(CH₃)CH₂CH₃ | CH₃ | CH(CH₃)₂ | CH₃ | 3 |
| E₂ | 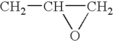 | H | CH(CH₃)₂ | CH₃ | CH(CH₃)₂ | CH₃ | 2 |
| Roseotoxin B | CH₂CH═CH₂ | CH₃ | CH(CH₃)CH₂CH₃ | CH₃ | CH(CH₃)₂ | CH₃ | 2 |
| Roseocardin | CH₂CH(CH₃)₂ | CH₃ | CH(CH₃)CH₂CH₃ | CH₃ | CH(CH₃)₂ | CH₃ | 2 |
| Desmethyldestruxin | CH₂CH(CH₃)₂ | H | CH(CH₃)CH₂CH₃ | H | CH(CH₃)₂ | CH₃ | 2 |
| B | | | | | | | |
| Chlorohydrin | CH₂CH(OH)CH₂Cl | H | CH(CH₃)CH₂CH₃ | CH₃ | CH(CH₃)₂ | CH₃ | 2 |
| Acetylchlorohydrin | CH₂CH(OCOCH₃)CH₂Cl | H | CH(CH₃)CH₂CH₃ | CH₃ | CH(CH₃)₂ | CH₃ | 2 |
| A₄ chlorohydrin | CH₂CH(OH)CH₂Cl | H | CH(CH₃)CH₂CH₃ | CH₃ | CH(CH₃)CH₂CH₃ | CH₃ | 2 |
| A₄ | CH₂CH═CH₂ | H | CH(CH₃)CH₂CH₃ | CH₃ | CH(CH₃)CH₂CH₃ | CH₃ | 2 |
| Homodestruxin B | CH₂CH(CH₃)₂ | H | CH(CH₃)CH₂CH₃ | CH₃ | CH(CH₃)CH₂CH₃ | CH₃ | 2 |
| Lac-6destruxin E | CH₃ | H | CH(CH₃)CH₂CH₃ | CH₃ | CH(CH₃)₂ | CH₃ | 2 |
| 2-hydroxy-4- | CH₂—C≡CH | H | CH(CH₃)CH₂CH₃ | CH₃ | CH(CH₃)₂ | CH₃ | 2 |
| pentinoic acid | | | | | | | |
| Protodestruxin | CH₂CH(CH₃)₂ | H | CH(CH₃)CH₂CH₃ | H | CH(CH₃)₂ | H | 2 |

In accordance with the present invention, the destruxin derivative represented by the above chemical formula I can be used as a preventive agent or a therapeutic agent of osteoporosis due to its strong inhibitory effect against osteoclasts. Accordingly, the present invention relates to a pharmaceutical composition containing a destruxin derivative represented by the chemical formula I and its pharmaceutically acceptable salt as an active ingredient.

Upon being used clinically, the pharmaceutical composition of the present invention may be, in combination with a suitable carrier easily selectable by those of ordinary skill in the art, formulated in tablets, capsules, troches, aqueous liquid solutions, suspensions, and the like for oral administration, in addition to, for injection, solutions and suspensions, as well as dried powders capable of being immediately applied, and the like, and may be also further formulated in liquid sprays, suppositories, transdermal patches, and the like.

The pharmaceutical composition prepared using the suitable carrier can be administered orally, or parenterally, for example, intravenously, transdermally, intraperitoneally, intranasally, buccally, and via other body cavities, or topically. In addition, the destruxin derivative according to the present invention may be typically administrated to a human in an amount of 0.1 μg to 100 mg, and preferably, 100 μg to 1 mg, and may be separately administrated according to a prescription typically several times per day at regular intervals, and preferably, one to six times per day.

In an embodiment of the present invention, with an aim to investigate an inhibitory effect of destruxin derivatives against induction of osteoporosis, assay methods for their inhibitory activity against bone resorption mediated by osteoclasts is achieved by evaluating their inhibition levels against bone resorption. An assay method, pit formation assay, is carried out by analyzing the number and volume of pits, which is a trace formed by bone resorption action of osteoclasts. The pit formation assay demonstrates that volume of pits is reduced parallel with the inhibitory effect of destruxin derivatives on osteoclastic bone resorption. Other assay method is to analyze actin rings, which function to support the cytoskeleton structure of osteoclasts, where inhibition of osteoclasts by destruxin derivatives results in abnormal actin rings.

The present invention will be explained in more detail with reference to the following examples in conjunction with the accompanying drawings. However, the following examples are provided only to illustrate the present invention, and the present invention is not limited to them.

### BRIEF DESCRIPTION OF THE DRAWINGS

FIG. 1 is a photograph showing mature osteoclasts which are TRAP (Tartrate-Resistant Acid Phospatase)-positive;

FIGS. 2A to 2D are photographs showing (A) normal osteoclasts, (B) pits formed by bone resorption of osteoclasts on the bone surface, (C) modified and shrinking morphologies of osteoclasts treated with destruxin E, and (D) reduction of pit size when treating osteoclasts with destruxin E;

FIGS. 3A to 3B are graphs showing (A) number of pit versus the concentration of destruxin E, and (B) normal structure of actin rings of osteoclasts treated with destruxin E in comparison with normal osteoclasts;

FIG. 4 is a graph showing that increased bone resorption of osteoclasts stimulated by parathyroid hormone (PTH) is reduced through action of destruxin E; and

FIGS. 5A and 5B are graphs showing an effect of destruxin E on release of ⁴⁵Ca in organ culture, in which (A) shows reduced release of ⁴⁵Ca versus increased destruxin E concentration in medium, and (B) shows an inhibitory effect of destruxin more effective than that of eel calcitonin.

### BEST MODES FOR CARRYING OUT THE INVENTION

### EXAMPLE 1

### Preparation of Osteoclasts

Osteoclasts were prepared through co-culture of osteoblasts and bone marrow cells, as will described below.

### Experimental Example 1

### Preparation of Osteoblasts

After sacrificing 50 one-day postnatal mice and cleaning them in 70% ethanol, calvarias were collected, transferred into 50 mL tubes having 10 mL α-MEM medium (Gibco, USA) containing 0.2% dispase (Boehringer Mannheim, Germany) and 0.1% collagenase (Wako Inc.), and then incubated at 37° C. for 5 minutes (mins) with shaking at 200 rpm. Thereafter, the medium was centrifuged and harvested, and calvarias remaining in the tubes were added again with 10 mL α-MEM medium containing 0.2% of dispase and 0.1% of collagenase, followed by incubation with shaking at 200 rpm at 37° C. for 10 mins. The incubation and harvesting of medium was repeated three times, giving 40 mL of medium containing osteoblasts. The 40 mL medium was then centrifuged at 1000 rpm for 5 mins, thereby osteoblasts were obtained as pellet, and the pellet was suspended in 10 mL 10% FCS (fetal calf serum)-containing α-MEM medium, and an additional 15 mL 10% FCS-containing α-MEM medium was added. From the 25 mL osteoblast suspension, 5 mL was put into each of five 100×20 mm culture dishes containing 10 mL α-MEM medium (10% FCS), and incubation was carried out at 37° C. under 5% CO₂. After incubation for 2 days, osteoblasts were observed and harvested. The harvesting of osteoblasts was accomplished by primarily removing medium, adding 0.2% collagenase-containing cc-NEM medium (10% FCS) to the culture dish, incubating in a 5% CO₂ incubator at 37° C. for about 20 mins, and then centrifuging the medium at 1000 rpm. The finally harvested osteoblasts were again suspended in α-MIEM medium (10% FCS) for use in preparing osteoclasts in the following Experimental Example 3.

### Experimental Example 2

### Preparation of Bone Marrow Cells

After sacrificing ddY mouse (6 to 9 weeks, male) by dislocation of spinal column and disinfecting in 70% ethanol, tibia and femur were obtained by removing attached muscle, cutting the central region of tibia, and then dislocating the knee joint. The ends of tibia and femur were cut, and 1 to 2 mL α-MEM (10% FCS) medium was injected into the inside of the bones and then sucked up using a 25 gage syringe, collecting bone marrow cells. Whole bone marrow cells were obtained from three ddY mice. Thereafter, centrifugation was performed at 1000 rpm to harvest cells, and the resulting pellet was suspended in 3 mL of 10 mM Tris HCl buffer solution (pH 7.5) containing 0.83% NH₄Cl to eliminate erythrocytes, followed by centrifugation at 1000 rpm, giving about 1×10⁷ bone marrow cells per tibia or femur. The obtained bone marrow cells were used in preparing osteoclasts in the following Experimental Example 3.

### Experimental Example 3

### Preparation of Osteoclasts

The formation and isolation of osteoclasts were accomplished on a culture dish coated with a collagen gel coating medium, prepared as follows: collagen gel, 5×α-MEM medium containing NaHCO₃, and 2.2% NaHCO₃ or 200 mM HEPES-containing 0.05 M NaOH buffer solution (pH7.4) were mixed in a ratio of 7:2:1 at low temperature, and 4 to 5 mL of the collagen gel medium was poured into a 100 mm culture dish and the dish was gently swirled to be form a uniform coating which gelatinized at 37° C. for 5 mins, and then stored at 4° C.

Osteoblasts (about 1×10⁵ cells) and bone marrow cells (about. 1×10⁷ cells) prepared in Experimental Examples 1 and 2, respectively, were co-cultured on the gelatinized medium for 6 to 8 days while fed with α-MEM medium (10% FCS) containing active vitamin D₃ (10⁻⁸ M), where the α-MEM medium was exchanged with new one once per 2 days. Thereafter, the culture medium was decanted, and 3 mL of α-MEM medium (without FCS) containing dispase and collagenase was added and incubated with shaking at about 300 rpm at 37° C. to dissolve collagen gel.

Using a polypropylene pipette having a wide inlet, cells were suspended, transferred to a 50 mL tube, and centrifuged at 200 rpm for 5 mins, and the resulting pellet containing osteoclasts was suspended in α-MEM medium (10% FCS).

Typically, osteoclasts have TRAP (Tartrate-Resistant Acid Phospatase) activity and large quantities of clacitonin receptors, as well as bone resorption properties. Therefore, in the embodiment of the present invention, TRAP staining was carried out to easily discriminate osteoclasts from other cells.

### EXAMPLE 2

### Identification of Osteoclast Formation

### Experimental Example 1

### Preparation of a Reaction Solution for TRAP (Tartrate-Resistant Acid Phospatase)

5 mg of a substrate, naphtol AS-MX phosphatase (Sigma, USA) was dissolved in 0.5 mL N,N-dimethylformamide, and 0.1 N NaHCO₃ buffer solution (pH 5) containing 50 mM tartarate (Sigma, USA) was added up to 50 mL volume, and 30 mg of pigment, Fast Red Violet LB salt (Sigma, USA), was then added and dissolved.

### Experimental Example 2

### TRAP Staining

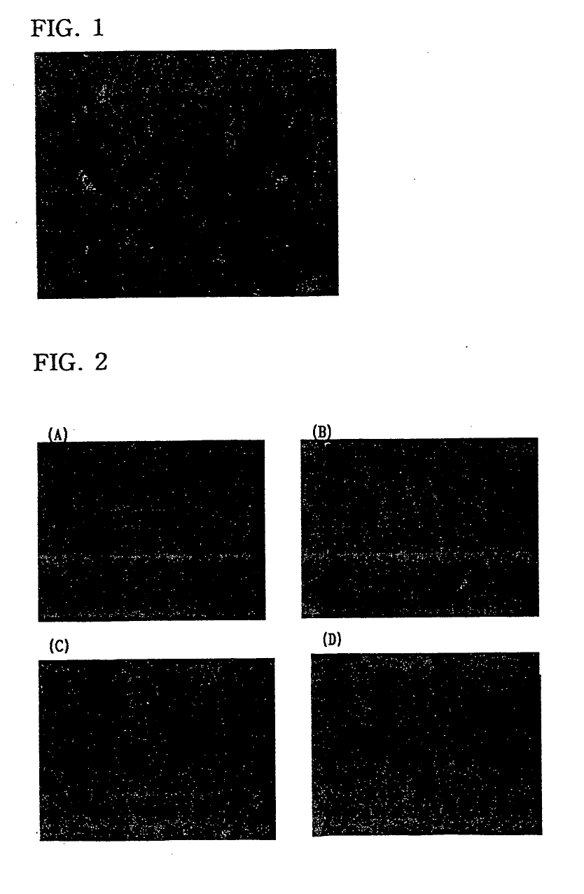

After culture medium was removed from a plate where osteoclasts had formed, the plate was fixed with 10% formalin for 5 to 10 mins and dried. The plate was again fixed with a solution of ethanol/acetone (1:1) and dried. After fixing, a TRAP solution was added to the plate, and the plate was left at room temperature for 10 to 15 mins. After removing the reaction solution, the plate was washed and dried, and observed microscopically. As shown in FIG. 1, large quantities of cells were TRAP-positive, demonstrating formation of large numbers of osteoclasts.

### EXAMPLE 3

### Test for Bone Resorption Ability of Osteoclasts

### Experimental Example 1

### Pit Formation Assay

Since most osteoclasts growing on bone surfaces have the ability to absorb bone, leaving pits, whether the osteoclasts prepared in Example 1 have bone resorption ability or was not determined by analyzing the number and volume of pits.

Bone fragments were prepared in a thickness of 1 mm, put in methanol, disinfected under UV light on a clean bench, and then transferred to wells of a 96-well plate, after which 100 μl α-MEM medium (10% FCS) was added. Bone fragments were treated with various concentrations of destruxin E, and 100 μl of the obtained osteoclast solution was added to each well, and the plate was gently swirled, followed by incubation at 37° C. under 5% CO₂. After incubation for 1 day, bone fragments and osteoclasts growing on them were observed micoscopically and then stored at 4° C.

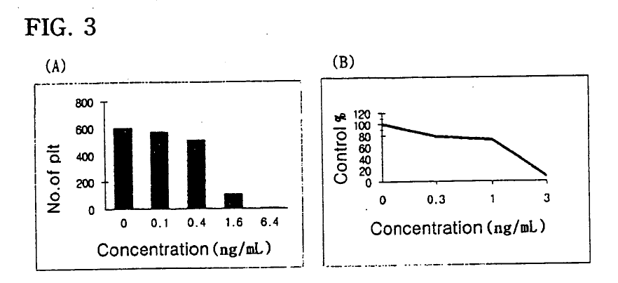

To analyze the number and volume of pits formed on the bone fragments under a microscope, keeping a portion where osteoclasts grew facing upward, the bone fragments were taken out from the 96 well plate, put on paper towel, and then stained with 6 μl of a hematoxin solution ((Sigma, USA). After 3 to 5 mins, the hematoxin solution was primarily removed and completely eliminated in an acidic solution, to microscopically investigate pits formed on the bone fragments. With reference to FIGS. 2A to 2D, normal osteoclasts are illustrated in FIG. 2A, and FIG. 2B shows pits on the bone surface, formed upon bone resorption by osteoclasts, and FIG. 2C shows modified and shrinking osteoclasts treated with destruxin E, and FIG. 2D shows reduction in pit volume upon treating osteoclasts with destruxin E. FIG. 3A shows the number of pits formed by osteoclasts treated with destruxin E and by osteoclasts not treated with destruxin E. As apparent in FIG. 3A, when the concentration of destruxin E was increased, the number of pits was reduced. In addition, as shown in Table 2, below, destruxin E was found to inhibit pit-forming activity of osteoclasts in a dose-dependent manner, and especially, 10 nM destruxin E inhibited pit formation by 50% (IC₅₀).

TABLE 2
Inhibition of pit formation by destruxin E

| | Conc. of destruxin E (nM) | | | | | | |
|---|---|---|---|---|---|---|---|
| | 0 | 0.5 | 1 | 5 | 10 | 50 | 100 |
| Inhibitory activity | 0 | 2 | 5 | 34 | 50 | 95 | 100 |
| against pit formation (%) | | | | | | | |

### Experimental Example 2

### Actin Ring Assay

In order to observe the formation and shape of actin rings, which function to support the cytoskeleton structure of osteoclasts, an actin ring assay was performed. After removing culture medium, osteoclasts cultured in the presence or absence of destruxin E were fixed with formalin for 10 mins, washed once with a solution containing Tween 20, and treated with 300 U rhodamine phalloidin (Sigma, USA) dissolved in methanol in a dark room. Intracellular distribution of actin rings was observed under UV light, and the result is given in Table 3, below. 50% of osteoclasts treated with 8 nM destruxin E (IC₅₀) were found to have abnormal actin ring structure. As apparent in FIG. 3B which is a graph showing degree of normality of actin rings of osteoclasts treated with destruxin E in comparison with degree of normality of actin rings of osteoclasts not treated with destruxin E, it was observed that the number of normal actin rings drops with increasing concentration of destruxin E.

TABLE 3
Inhibitory activity of destruxin E against
actin ring formation

| | Conc. of destruxin E (nM) | | | | | | |
|---|---|---|---|---|---|---|---|
| | 0 | 0.5 | 1 | 5 | 10 | 50 | 100 |
| Inhibitory activity against | 0 | 1 | 3 | 10 | 65 | 100 | 100 |
| actin ring (%) | | | | | | | |

### EXAMPLE 4

### Test for Inhibitory Activity of Destruxin E Against ⁴⁵Ca Release Through Mouse Organ Culture

15 to 16 days pregnant ddY mice were injected subtaneously with a solution of ⁴⁵Ca-labelled calcium chloride to label their bone. After one day, mice were anesthetized with ether, and disinfected in 70% ethanol. Fetuses were obtained from, and transferred to a sterile dish. Under a stereo microscope, after cutting forelegs and peeling off skin thereof, attached muscle was removed from radius and ulna, and cartilage portions existing at the both ends of the two bones were then obtained. During the preparation, the bones were kept moist. Before culturing, a stainless steel membrane was put into a 24-well plate containing 0.5 mL BGJ_{b} medium (Gibco, USA), and the obtained bone was then placed onto the membrane. After incubation for 24 hours, bone was transferred to destruxin E-containing medium and further incubated for 72 hours. Herein, parathyroid hormone (PTH) inducing release of calcium was used, and the inhibitory effect of destruxin E on the calcium release by PTH was analyzed. The amounts of ⁴⁵Ca remaining in bone and released to medium were measured using 5% TCA, and bone-resorbing activity was calculated as follows.

Bone-resorbing activity=released amount of ⁴⁵Ca to medium/(released amount of ⁴⁵Ca to medium+amount of ⁴⁵Ca remaining in bone)×100

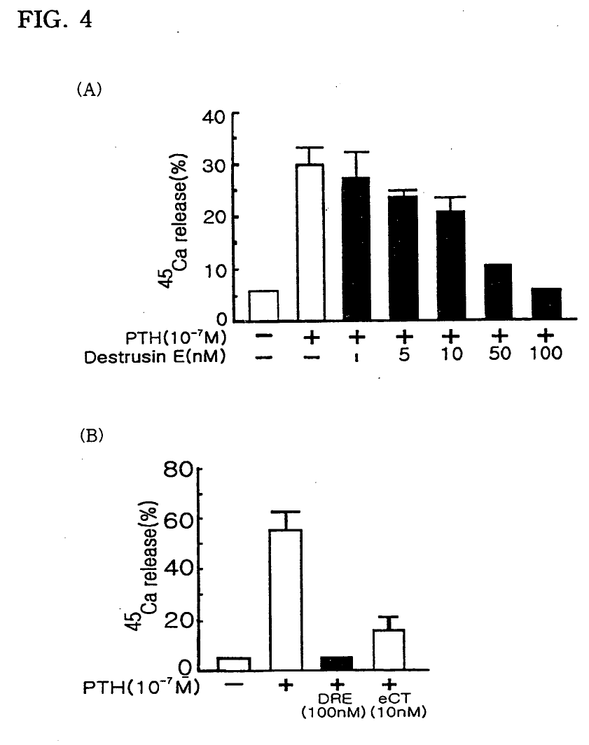

With reference to FIG. 4A, which illustrates the inhibitory effect of destruxin E on the release of ⁴⁵Ca in a dose-dependent manner, it was observed that, when the concentration of destruxin E is increased, the release amount of ⁴⁵Ca is reduced. In addition, as apparent in FIG. 4B showing comparison of inhibitory effect of destruxin E with that of eel calcitonin, the inhibitory effect of destruxin E is higher than that of eel calcitonin.

### EXAMPLE 5

### Inhibitory Activity of Destruxin B Against Osteoclasts

A test for measuring inhibitory activity of destruxin B against osteoclasts was performed according to the same method as in Example 4. As shown in Table 4, below, 50% inhibition (IC₅₀) of pit formation was observed at 0.2 μM of desrruxin B, and 50% inhibition (IC₅₀) of actin ring formation at 0.6 μM of destraxin B, demonstrating that inhibitory activity of destruxin B is very excellent, although the inhibitory activity of destruxin B is lower than that of destruxin E.

TABLE 4
Inhibitory activity of destruxin B against osteoclasts

| | Conc. of destruxin | | | | |
|---|---|---|---|---|---|
| | B (μM) | | | | |
| | 0 | 0.1 | 0.5 | 1 | 5 |
| Inhibitory Activity versus pit formation | 0 | 25 | 71 | 94 | 100 |
| Inhibitory Activity versus actin ring (%) | 0 | 12 | 41 | 92 | 100 |

### EXAMPLE 6

### Effect of Destruxin A, B, and E on the Morphology and Survival of Osteoclasts

Osteoclasts were treated with destruxin derivatives, A, B, and E, and then ED₅₀ (concentration at which morphological changes are observed in 50% of osteoclasts) was measured and MIC (Minimum Inhibitory Concentration at which survival of all osteoclasts is inhibited), and the results are given in Table 5, below. As apparent in Table 5, destruxin A showed ED₅₀ and MIC of 0.2 μM and 20 μM, respectively, and destruxin B showed ED₅₀ and MIC of 0.3 μM and 10 μM, respectively, and destruxin E showed ED₅₀ and MIC of 0.02 μM and 0.2 μM, respectively, indicating that destruxin E has the highest inhibitory effect against osteoclasts.

TABLE 5
Effect of destruxin derivatives on the morphology and survival
of osteoclasts

| | ED₅₀ | MIC |
|---|---|---|
| | (μM) | (μM) |
| Destruxin A | 0.2 | 20 |
| Destruxin B | 0.3 | 10 |
| Destruxin E | 0.02 | 0.2 |

[Note]
ED₅₀: Concentration at which morphological changes are seen in 50% of osteoclasts.
MIC: Minimum Inhibitory Concentration at which survival of all osteoclasts is inhibited.

### INDUSTRIAL APPLICABILITY

As described above in examples and experimental examples, according to the present invention, destruxin derivatives have an excellent inhibitory effect on pit formation and actin ring formation in osteoclasts, leading to inhibition of osteoclastic bone resorption. Therefore, the pharmaceutical composition containing a destruxin derivative of the present invention is very useful for the prevention and treatment of osteoporosis.

1. A pharmaceutical composition for the prevention and treatment of osteoporosis containing a destruxin derivative as an active ingredient, represented by the following chemical formula I: 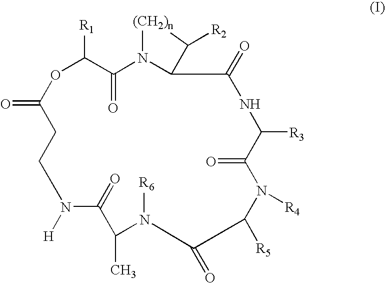
wherein, R₁ group is CH₃, CH₂—CH═CH₂, CH₂CH(CH₃)₂, CH₂CH(CH₃)CH₂OH, 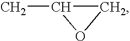
CH₂CH(CH₃)COOH, CH₂CH(OH)CH₂Cl, CH₂—C≡CH, or CH₂CH(OCOCH₃)CH₂Cl; R₂ , R₄ and R₆, which may be the same or different, are H or CH₃; R₃ and R₅, which may be the same or different, are CH(CH₃)CH₂CH₃ or CH(CH₃)₂; and n is 2 or 3.

2. The pharmaceutical composition as set forth in claim 1, wherein the destruxin derivative is destruxin E.

3. The pharmaceutical composition as set forth in claim 1, wherein the destruxin derivative is destruxin B.

4. The pharmaceutical composition as set forth in claim 1, wherein the destruxin derivative is destruxin A.
